(19) United States
(12) Patent Application Publication
(10) Pub. No.: US 2022/0072540 A1
(43) Pub. Date: Mar. 10, 2022

(54) MICROFLUIDIC DEVICES

(71) Applicant: Hewlett-Packard Development Company, L.P., Spring, TX (US)

(72) Inventors: Silam J. Choy, Corvallis, OR (US); Hilary Ely, Corvallis, OR (US)

(73) Assignee: Hewlett-Packard Development Company, L.P., Spring, TX (US)

(21) Appl. No.: 17/417,362

(22) PCT Filed: May 15, 2019
(86) PCT No.: PCT/US2019/032457

§ 371 Date: Jun. 22, 2021

(51) Int. Cl. B01L 3/00; B01L 7/00

(52) U.S. Cl. CPC B01L 3/502715; B01L 3/502707; B01L 2200/16; B01L 2300/16; B01L 7/52

(57) ABSTRACT

A microfluidic device includes a semiconductor microchip including fluid active circuitry and transistor circuitry, wherein the transistor circuitry provides onboard logic at the semiconductor microchip to control the fluid active circuitry. The microfluidic device further includes a microfluidic chamber fluidly coupled to an inlet port and an outlet port, wherein the microfluidic chamber is defined in part by a microchip surface with the active circuitry positioned to interact with fluid introduced into the microfluidic chamber and partially defined by an enclosing surface. The microchip surface, the enclosing surface, or both include a chemically-modified microfluidic chamber surface that is selectively interactive with a target component of the fluid.

### BACKGROUND

Microfluidic devices can exploit chemical and physical properties of fluids on a microscale. These devices can be used for research, medical, and forensic applications, to name a few, to evaluate or analyze fluids using very small quantities of sample and/or reagent to interact with the sample than would otherwise be used with full-scale analysis devices or systems.

### BRIEF DESCRIPTION OF THE DRAWINGS

FIG. 1 graphically illustrates a schematic cross-sectional view of an example microfluidic device in accordance with the present disclosure;

FIG. 2A graphically illustrates a schematic perspective view of a portion of a microfluidic device in accordance with the present disclosure;

FIG. 2B graphically illustrates a schematic perspective view of a portion of alternative microfluidic device in accordance with the present disclosure;

FIG. 3 graphically illustrates a schematic cross-sectional view of an example microfluidic device in accordance with the present disclosure;

FIG. 4 graphically illustrates a schematic cross-sectional view of an example microfluidic device in accordance with the present disclosure;

FIG. 5 graphically illustrates a schematic cross-sectional view of an example microfluidic device in accordance with the present disclosure;

FIG. 6 graphically illustrates a schematic cross-sectional view of an example microfluidic device in accordance with the present disclosure;

FIG. 7 is a flow diagram illustrating an example method of manufacturing a microfluidic device in accordance with the present disclosure; and

FIG. 8 is a flow diagram illustrating an example method of electronically interacting with a target substance of a fluid in accordance with the present disclosure.

### DETAILED DESCRIPTION

With microfluidics, the isolation or concentrating of target substances that may be dissolved or dispersed in a fluid can provide benefits with respect to subsequent processing, including measurement, chemical reaction or interaction, physical manipulation, or the like. Once isolated or concentrated, these or other subsequent processes can occur in situ within the microfluidics of a semiconductor microchip, for example. To illustrate, measurements can be performed using sensors on the semiconductor microchips, chemical reactions or interactions can be initiated within reaction chambers or microchannels, local heating can be carried out, physical fluidic or target substance manipulation can occur using MEMS components, etc. In further detail, surfaces that define the microfluidic chamber can be chemically-modified to interact with components of fluids that are introduced and/or passed through the microfluidic chamber to be used in conjunction with fluid active circuitry or a semiconductor microchip. Thus, both a chemically-modified microfluidic chamber surface and the fluid active circuitry can interact with the fluid within the chamber to process the fluid as may be useful for a particular application. For example, chemically-modified microfluidic chamber surfaces can include chemistry capable of selectively capturing a target substance from a fluid, such as a biological fluid. Target substances may include, for example, biological cells, proteins, nucleic acids, exosomes, and other compounds or particles. In some examples, unwanted components (other than the target substance) could be purged from the microfluidic chamber without much loss of the target substance, e.g., the target substance can become more concentrated within the microfluidic chamber.

In accordance with this example and others, the present disclosure is drawn to a microfluidic device including a semiconductor microchip including fluid active circuitry and transistor circuitry, wherein the transistor circuitry provides onboard logic at the semiconductor microchip to control the fluid active circuitry. The microfluidic device also includes a microfluidic chamber fluidly coupled to an inlet port and an outlet port, wherein the microfluidic chamber is defined in part by a microchip surface with the active circuitry positioned to interact with fluid introduced into the microfluidic chamber and partially defined by an enclosing surface. The microchip surface, the enclosing surface, or both is a chemically-modified microfluidic chamber surface that is selectively interactive with a target component of the fluid in this example. In further detail, the microfluidic device can further include the fluid containing the target fluid, and the fluid can be positioned or loaded within the microfluidic chamber. In another example, the enclosing surface can be provided by a lid including a material selected from glass, quartz, polymer, amorphous polymer, or a combination thereof. In still other examples, the enclosing surface can be provided by a support substrate that supports the semiconductor microchip. The support substrate can include a material selected from metal, glass, silicon, silicon dioxide, ceramic, polyethylene, polypropylene, polycarbonate, poly(methyl methacrylate), epoxy molding compound, polyamide, liquid crystal polymer (LCP), polyphenylene sulfide, or a combination thereof. The chemically-modified microfluidic chamber surface can be chemically-modified with an antibody, streptavidin, an oligomer, an amine-containing functional group, a carboxyl-containing functional group, an organosilane, or a combination thereof. The semiconductor microchip can, in some examples, have an elongated aspect ratio with a width from 50 μm to 1 mm, a thickness from 50 μm to 1 mm, and a length of 1.5 mm to 50 mm. The inlet port and the outlet port can be positioned so that a flow of fluid therebetween is along the length of the semiconductor microchip. In another example, the microfluidic chamber can have a volume from 1 nL to 100 μL. The fluid active circuitry can include, as an example, a heater, a sensor, an electromagnetic radiation source, a fluid actuator, or a combination thereof.

In another example, a method of making a microfluidic device includes forming a microfluidic chamber fluidly coupled to an inlet port and an outlet port that is partially defined by a microchip surface of a semiconductor microchip and partially defined by an enclosing surface, wherein the semiconductor microchip includes fluid active circuitry and transistor circuitry. The transistor circuitry in this example provides onboard logic at the semiconductor microchip to control the fluid active circuitry. The method further includes chemically modifying the microchip surface, the enclosing surface, or both to form a chemically-modified microfluidic chamber surface. In one example, chemically modifying the microchip surface, the enclosing surface, or both can occur prior to assembly of the microfluidic chamber. In another example, chemically modifying the microchip surface, the enclosing surface, or both can occur after assembly of the microfluidic chamber.

In another example, a method of electronically interacting with a target substance of a fluid includes flowing a fluid that includes a target substance through a microfluidic chamber which includes a chemically-modified microfluidic chamber surface, wherein the microfluidic chamber is partially defined by a semiconductor microchip that includes transistor circuitry and fluid actionable circuitry. The method further includes selectively retaining the target substance at the chemically-modified microfluidic chamber surface while allowing secondary fluid components to exit the microfluidic chamber, and electronically inducing an interaction between the target substance with the semiconductor microchip using the transistor circuitry to provide onboard logic to operate the fluid active circuitry. The target substance can, for example, be a nucleic acid, and the secondary fluid components can include lysed cellular debris. In further detail, the onboard logic can control multiple operations of the fluid active circuitry based on conditions within the microfluidic chamber by selecting operations from multiple alternatives.

In addition to the examples described above, the microfluidic device, the method of manufacturing the microfluidic device, and the method of electronically interacting with a target substance of a fluid are described in greater detail below. It is also noted that when discussing the microfluidic device or one or both methods, such discussions of one example are to be considered applicable to the other examples, whether or not they are explicitly discussed in the context of that example. Thus, in discussing solids supports in the context of the microfluidic device, such disclosure is also relevant to and directly supported in the context of the methods, and vice versa.

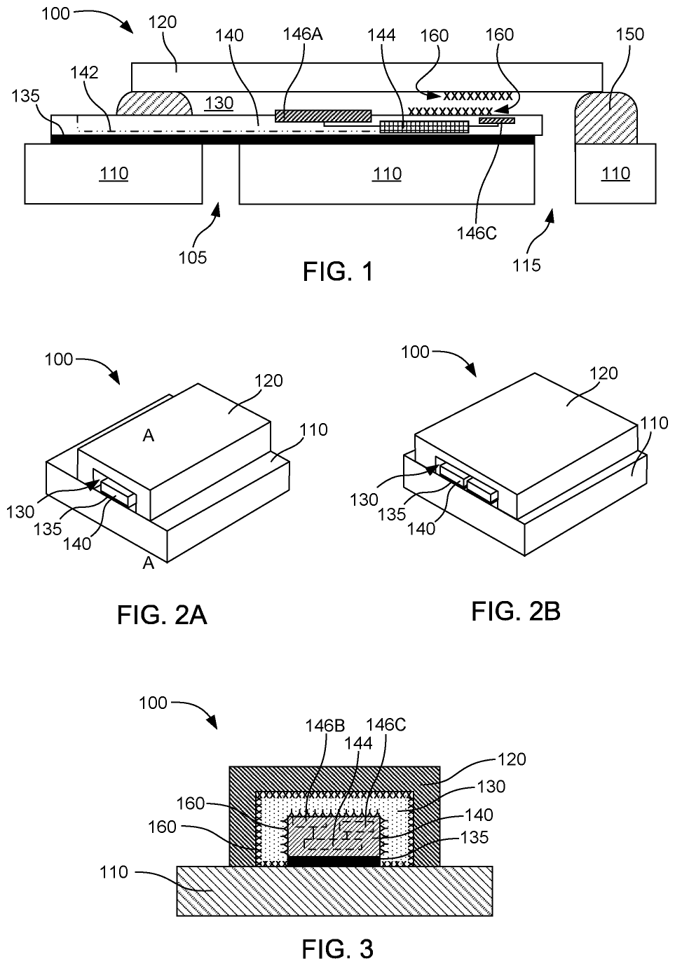

Turning now to the FIGS. for further detail, as an initial matter, there are several components of the microfluidic devices shown that are common to multiple examples, and thus, the common reference numerals are used to describe various features. Thus, a general description of a feature in the context of a specific FIG. can be relevant to the other example FIGS. shown, and as a result, individual components need not be described and then re-described in context of another FIG. In the following example descriptions, FIGS. 1-6 can be considered simultaneously in the description of the FIGS. to the extent relevant by a common reference numeral, for example.

In further detail, the representations of the microfluidic devices in the figures are merely intended to facilitate the description and presentation of the microfluidic devices disclosed herein. It is noted, however, that when discussing microfluidic devices, methods, or the like, such description is also intended to encompass mesofluidic devices. Thus, in some examples, the microfluidic chambers can include sub-millimeter dimensions. In other examples, the microfluidic chambers can include from millimeter to centimeter dimensions. Thus, for simplicity, both microfluidics and mesofluidics are referred to herein as microfluidics, meaning generally what is being referred to are fluidics that use small quantities of fluids in the chambers, e.g., 1 nL to 100 μL, from 100 nL to 1 μL, from 1 μL to about 10 μL, or from 500 nL to 6 μL.

With more specific reference to FIG. 1 (and other FIGS. with common features), a schematic cross-sectional view of an example microfluidic device 100 in accordance with the present disclosure is shown. As shown, the microfluidic device includes a support substrate 110 with an inlet port 105 and an outlet port 115. The inlet port and the outlet port can be used to provide fluid to (via the inlet port) and pass fluid from (via the outlet port) a microfluidic chamber 130. It is noted that the terms “inlet” and “outlet” do not infer that these ports interact with the microfluidic chamber in one direction, though that could be the case. In some instances, there may be occasion for the fluid to flow “backwards” or “bidirectionally,” and thus the terms “inlet port” and “outlet port” are used because at some point during operation, these two ports act as inflow of fluid and outflow of fluid, respectively, relative to the microfluidic chamber.

In further detail, the microfluidic device 100 includes a semiconductor microchip 140 with transistor circuitry 144 that can use onboard logic to control fluid active circuitry 146A-1460, which can be in the form of any of a number of fluid active circuitry devices, such as fluid active circuit devices that operate as a heater (e.g., rapid thermal cycling heater, resistive heater, etc.), a sensor (e.g., photo sensor, thermal sensor, fluid flow sensor, chemical sensor, etc.), an electromagnetic radiation source (photo diode, laser, etc.), a fluid actuator (e.g., mixers, bubblers, pumps, etc.), or the like. A semiconductor microchip can operate with fewer electrical connections then other types of circuitry systems, and thus, a single or few data lines 142 may be used to operate multiple circuitry components, rather than having a separate conductive pad(s) and control trace for every fluid active circuit that may be present. In other words, with onboard logic provided by the transistor circuitry, there can be a reduced number of I/O ports present. This is because the operation can be controlled partially or fully by the transistor circuitry operating as a logic control for the fluid active circuitry. The transistor circuitry 144 may include an integrated circuit to perform the operations according to logical relationships or state transitions implemented in the transistor circuitry 144. The transistor circuitry 144 may include an application specific integrated circuit (ASIC), a Field Programmable Gate Array (FPGA), or the like.

In addition to the transistor circuitry 144, as mentioned, the semiconductor microchip 140 includes fluid active circuitry 146A-1460. In the various examples shown herein, there are multiple fluid active circuitry components shown in the various FIGS., but there could be fewer or more and/or there could be arrays of fluid active circuitry, etc. For example, there can be a single fluid active circuit, an array or one type of fluid active circuit, multiple types of fluid active circuits, arrays of multiple types of fluid active circuits, or any combination thereof. Particularly when there are multiple fluid active circuits present (by type, by array, and/or a single type), or when there are decisions to be made in the operation of the fluid active circuitry that would benefit from speed or efficiency, the use of an onboard transistor to provide logic control to the fluid active circuitry can be beneficial. For example, if an operation would benefit from maintaining a fluid within a narrow temperature range, a heater circuit or cooling functions can be used to keep the temperature with that narrow temperature range, e.g., within 1° C., within 0.5° C., or within 0.1° C., without sending signals on and off the semiconductor microchip to receive “decisions” from a CPU that is not part of the semiconductor microchip. Thus, by increasing response time to adjust temperature using onboard logic, the temperature may be able to be kept within a narrower target temperature range.

In some examples, the fluid active circuitry 146A-146C can be in physical contact with a fluid when fluid is introduced into the microfluidic chamber, or there may be a thin protective film or layer of material that protects the circuitry, but which does not interfere the function of the active circuitry in interacting with a fluid or target substance of a fluid. For example, there may be a protective film(s) or layer(s) of polymer, oxide, carbide, metal or alloy, nitride, silicon, etc. The thickness of a protective film(s) or layer(s) that may be included may range from the thickness of 0.3 nm (a single atom layer) to 50 μm, from 1 nm to 50 μm, from 10 nm to 40 μm, from 10 nm to 30 μm, from 10 nm to 1 μm, from 50 nm to 50 μm, from 50 nm to 30 μm, from 100 nm to 50 μm, from 500 nm to 50 μm, from 1 μm to 50 μm, from 5 μm to 50 μm, from 10 μm to 50 μm, from 1 μm to 30 μm, from 1 μm to 10 μm, from 1 nm to 500 nm, or from 1 nm to 200 nm, for example. These thicknesses tend to be thin enough that the fluid active circuitry can interact with the fluid or the target substance therein.

In further detail, the fluid active circuitry 146A-1460 (coated or uncoated) can protrude into the microfluidic chamber (as shown at 146A), can be positioned along a surface that defines the microfluidic chamber (as shown at 146B), or can be beneath a surface of the semiconductor microchip (as shown at 146C), for example, or can be positioned just below a surface of the semiconductor microchip. As shown in this particular example, a portion of the semiconductor microchip is attached to the support substrate via an adhesive 135, for example, but could be attached, suspended, cantilevered, or included therein in any of a number of other ways.

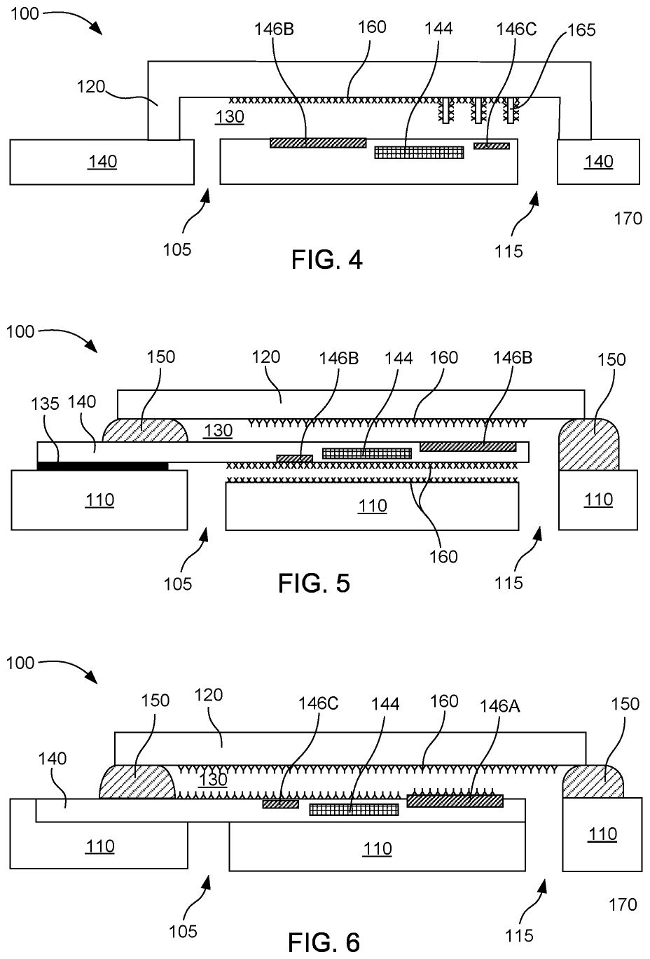

As shown by way of example, the microfluidic chamber 130 includes a chemically-modified microfluidic chamber surface 160. For example, the chemically-modified microfluidic chamber surface(s) could include any structure that defines the microfluidic chamber and which is non-transitory or mobile within the chamber, such as interior (chamber-facing) surfaces of the lid 120 (See FIGS. 1 and 3-6), the semiconductor microchip 140 (see FIGS. 1, 3, 5, and 6), or the support substrate 110 (see FIGS. 3 and 5). Other structures that may be present that can also be chemically modified include pillars (See 165 at FIG. 4), wall protrusions, surface bumps or processes, surface cavities, or the like, which may be attached or extensions of any of the surfaces defining the microfluidic chamber. In one example as shown in FIG. 5, the semiconductor microchip 140 is suspended above the substrate, and thus, the surface modification 160 can be present both upper and lower opposite-facing major surfaces, as well as on the support substrate where the semiconductor microchip might otherwise have been attached. This is one way of increasing the surface area within the microfluidic chamber where chemically-modified surfaces can be located. The term “major surface” is defined one or both or two surfaces that have the largest surface area. If a semiconductor microchip is adhered to a support substrate or embedded in a support substrate, only one “major” surface is exposed to the interior of the microfluidic chamber (See FIGS. 1-3 as an example). If the semiconductor chip is suspended, e.g., cantilevered, bridged, etc., then there may be two major surfaces that are exposed to the interior of the microfluidic chamber (See FIG. 5 as an example).

Examples of surface modifications that can be used include modification of the surfaces with covalent attached ligands, such as with antibodies, streptavidin, oligomers, e.g., sequence specific oligomers, functional groups including amines and/or carboxyl groups, or the like. In further detail, organosilanes or other linking groups having functional groups appended thereto can be used. In some examples, the chemically-modified surface can include functional moieties selective for nucleic acids or even a specific base or nucleic acid sequence. Notably, there can also be multiple types of surface modification chemistries in a common microfluidic chamber. FIGS. 3 and 5 show examples where there are two different types of chemically-modified surfaces, as schematically represented by different by an “x” shape and a “y” shape.

In further detail, the microfluidic chamber 130 can be defined on multiple sides by multiple structures. For example, the microfluidic chamber can be defined by a portion of a surface of the semiconductor microchip 140, including fluid active circuitry 146A-1460 thereof. A lid 120 and a seal 150 can also further define the microfluidic chamber. FIGS. 2A, 2B, 3 and 5 also show that the support substrate 110 further defines the microfluidic chamber. The lid, the support substrate, or other structures other than the semiconductor microchip can provide an “enclosing surface” to complement the microchip surface to define the microfluidic chamber. As a further note, the microfluidic chamber can have a larger cross-sectional area than the inlet port 105 or the outlet port 115, or in some examples, it is notable that the microfluidic chamber can sometimes have a smaller cross-sectional area than the inlet and/or outlet port. The cross-sectional area can be defined as the area that is perpendicular to fluid flow when the microfluidic device is in operation.

A variety of suitable support substrates 110 can be used. Typically, any support substrate to which a semiconductor microchip 140 (or a semiconductor microchip and a lid 120 in some examples) can be mounted, and that is suitable for a particular application, can be used. In some specific examples, the support substrate can include or be made of a material such as metal, glass, silicon, silicon dioxide, a ceramic material (e.g., alumina, aluminum borosilicate, etc.), a polymer material (e.g., polyethylene, polypropylene, polycarbonate, poly(methyl methacrylate), epoxy molding compound, polyamide, liquid crystal polymer (LCP), polyphenylene sulfide, etc.), the like, or a combination thereof. Additionally, the support substrate can have any suitable dimensions for a given application. In some examples, the support substrate and the lid can be architecturally compatible to form a complete seal at their interface. In other examples, the support substrate and the lid can be architecturally compatible so that a seal 150, such as a sealing adhesive, can be positioned between the support substrate and the lid to form or enclose the microfluidic chamber 130. Other arrangements that also use a support substrate can likewise be used, such as support substrates that support a cantilevered or suspended (bridge-like) semiconductor microchip within the microfluidic chamber, as shown by example in FIG. 5. In that example, the support substrate supports the semiconductor microchip just outside of the microfluidic chamber at one end using an adhesive 135, and the support substrate supports the semiconductor microchip at another end within the microfluidic chamber also using an adhesive, thereby forming a bridged semiconductor microchip with microfluidic chamber with space both above and below the semiconductor microchip. In this arrangement, there may be fluid active circuitry (not shown in FIG. 5, but shown by example in FIG. 1) on both sides of the semiconductor microchip.

The semiconductor microchip 140 can be any configuration that is suitable for performing a function as described herein. The semiconductor microchip can be a CMOS semiconductor microchip, for example. Furthermore, in addition to silicon-based semiconductor microchips, the semiconductor microchip can be of gallium arsenide or gallium. In one example, the semiconductor microchip can be an elongated semiconductor microchip. By “elongated semiconductor microchip,” it is to be understood that the semiconductor microchip can have a width to length where the width is narrower than the length. Example aspect ratios include width to length ratios such as 1:10 to 1:200, from 1:10 to 1:150, 1:10 to 1:100, from 1:10 to 1:50, or from 1:20 to 1:00, for example. There is also a thickness component to the ratio. Thickness for the semiconductor microchip can vary, but can be thin enough to leave space in the microfluidic chamber to allow for fluid flow through the microfluidic chamber and in communication with the fluid active circuitry.

In examples herein, a top surface (or portion thereof) defined by the length and width can be in contact with fluid within the microfluidic chamber, but in some examples, there can also be sides of the semiconductor microchip or a bottom surface of the semiconductor microchip that can be in contact with the fluid as well (see FIGS. 5 and 8, for example). It is noted that in referring to a structure using a term such as “top,” “side,” or “bottom,” these are considered to be relative terms that do not infer orientation, as the devices can be used in any orientation. Thus, the term “top” for example, is a term indicating location or a surface relative to a support substrate structure to which the semiconductor chip is supported in several of the example FIGS. As another example, if positioned vertically, the support substrate and another structure, such as a lid, would be positioned side by side. But the lid is shown in the FIGS. as being on “top” of the support substrate. Thus, in this context, as orientation is not inferred, the term “top” and other terms should be considered to be relative terms in the context of the FIGS.

The length of the semiconductor microchip can be, for example, from 1.5 mm to 50 mm, from 5 mm to 50 mm, from 10 mm to 40 mm, from 10 mm to 30 mm, from 15 mm to 50 mm, from 20 mm to 50 mm, or from 15 mm to 40 mm, for example. The width of the semiconductor microchip can be, for example, from 50 μm to 1 mm, from 100 μm to 1 mm, from 200 μm to 1 mm, from 500 μm to 1 mm, from 200 μm to 800 μm, or from 300 μm to 700 μm, for example. The thickness of the semiconductor microchip can be, for example, from 50 μm to 1 mm, from 100 μm to 1 mm, from 200 μm to 1 mm, from 500 μm to 1 mm, from 200 μm to 800 μm, or from 300 μm to 700 μm, for example.

In other examples, wherein the semiconductor microchip is not an elongated semiconductor microchip, the shape of the semiconductor microchip can be rectangular (including square), elliptical, circular, arcuate, polygonal, trapezoidal, or any other geometric shape. In further detail, the semiconductor microchip can be made of a variety of support materials, such as silicon, glass, quartz, ceramic, or the like. The fluid active circuitry can be in electrical communication with circuitry or other components outside of the microfluidic chamber via a wire, a trace, a network of wires, a network of traces, an electrode, a conductive pad, and/or any other electrical communication structure that may or may not be embedded in the semiconductor microchip support material. The fluid active circuitry that is included as part of the semiconductor microchip and which is in fluid communication with the microfluidic chamber (to interact in contact with fluid or interact with fluid beneath a thin film(s) or layer(s) on the semiconductor microchip) can be in the form of any of a number of fluid active circuitry components, including heaters, sensors, electromagnetic radiation sources, fluid actuators (e.g., mixers, bubblers, fluid pumps, etc.). In some examples, there can be multiple different types of fluid active circuitry components, e.g. a heater for rapid thermal cycling and a sensor to confirm something occurring within the microfluidic chamber. For example, a heater can be used for rapid thermal cycling to amplify DNA, and a sensor can be present to confirm the temperature profile and/or the presence of amplified DNA. Other combinations can be designed to work for a variety of specific purposes. Regardless, the fluid active circuitry described herein includes circuitry that is positioned to interact with the fluid(s) which flow into or through the microfluidic chamber, either with direct contact or protected by a thin film(s) or layer(s) of protective material, depending on the active circuitry materials and function.

Regarding the lid 120 (or cover), the lid can be any configuration that us usable for contributing to forming the microfluidic chamber. For example, the lid can have a “U-shape” as shown in FIG. 2A, FIG. 2B, and FIG. 3. Alternatively, the lid can have a flat shape, with walls provided by a separate wall structure. In either case, the lid can be fitted to attach to the support substrate 110, as shown in FIGS. 2A and 2B, to both the support substrate at some locations and to the semiconductor microchip 140 at other locations, as shown in FIGS. 1, 5, and 6, or to the semiconductor microchip, as shown in FIG. 4. Alternatively, the lid may have a more complicated shape or configuration, such as shaped to provide multiple discrete microfluidic chambers between semiconductor microchips, fluid active circuitry thereon, or other structures. In another example, the microfluidic device can include a second lid that can form a second discrete microfluidic chamber between semiconductor microchips or fluid active circuitry of semiconductor microchips. Furthermore, as shown in the example FIGS. herein, the various inlet ports 105 and the outlet ports 115 are shown as being provided by the support substrate and/or semiconductor microchip. However, it is understood that the inlet and/or outlet port can likewise be provided by the lid, for example. The positioning of the inlet port and/or outlet port is not particularly limited, except that the inlet port and the outlet port can be positioned so that fluid flow (at some point in time) flows through the microfluidic chamber. In further detail, the lid may provide other ports, such as vents or other structures for facilitating fluid flow through the microfluidic chamber.

The lid can be prepared or selected from materials, such as glass, quartz, metal, polymer, e.g., amorphous polymer, or other suitable materials. Non-limiting examples of polymers can include polydimethylsiloxane (PDMS), cyclic olefin polymer (COP), cyclic olefin copolymer (COC), polyethylene terephthalate (PET), and/or the like. In some examples, the lid can include or be made of a transparent or translucent material such as glass, quartz, polycarbonate, Trivex™ (PPG Industries, USA), cyclic olefin copolymer (COC), and/or the like. In some examples, the lid can include or be made of a non-translucent material, such as silicon, a metal, and/or the like. In some examples, the material used to manufacture the lid can be doped with a dopant to enhance thermal performance, optical performance, chemical performance, and/or the like. Non-limiting examples of dopants can include erbium, AlOₓ, TaOₓ, etc. Composites of multiple materials can likewise be used to form the lid.

In some examples, with respect to the lid dimension, referring to the largest lid surface structure or lid “top,” e.g., typically the lid surface opposite the support substrate, the structure of that portion of the lid can have dimensions that may be larger than the dimensions of a portion of the semiconductor microchip that is within the microfluidic chamber, for example, though this may not be the case in all instances.

Regarding the thickness of the lid 120 in particular, the thickness can vary depending on the particular application for which the microfluidic device 100 may be used. In some examples, the lid can have a thickness of from 0.1 mm to 10 mm, from 0.1 mm to 5 mm, from 0.2 mm to 2.5 mm, from 0.5 mm to 5 mm, or from 0.3 mm to 2 mm, for example. Thicknesses outside of this range can likewise be used, particularly if the thickness of the lid is not involved in a specific function that benefits from the lid thickness. For example, in some examples, the lid can be designed to be relatively thin to provide a function for a given application, such as to provide acceptable optical clarity (depending on the material, etc.), to provide acceptable heat dissipation from the microfluidic chamber 130, etc. Where a thinner lid is used, the lid can have a thickness of from 0.1 mm to 1 mm, or from 0.1 mm to 0.5 mm, for example. In other examples, a thicker lid may provide a desired property, such as decreased optical clarity or translucence, increased thermal insulation, etc. (as compared to a thinner lid). Where a thicker lid may be selected for use, the lid can generally have a thickness of from 0.5 mm to 10 mm, from 1 mm to 10 mm, or from 1 mm to 5 mm, for example. Further still, in some examples, it can be desirable to have a lid with a “non-uniform thickness” along one or more of the surfaces, e.g., the surface opposite the support substrate or top surface of the lid, as shown in FIG. 1 by example. A lid with a non-uniform thickness would include lids with thickness differentials that are not merely artifacts of manufacturing processes, but affirmatively designed lids with thickness differentials at various locations, e.g., two thicknesses at two locations that are coplanar (top portion of lid with two thicknesses), a sidewall thickness that is different than the top portion thickness, etc. This can be done for any of a number of reasons, such as to achieve structural, mechanical, functional, or other properties related to the lid structure. In further detail, the lid can be formed in a variety of ways, such as by injection molding, cast molding, compression molding, etching, cutting, melting, drilling, routing, and/or the like.

There are various structures that can be bonded or sealed together, and such bonding or formation of seals can be carried out using any of a number of technologies. To illustrate, in order to manufacture a microfluidic device 100, the semiconductor microchip 140 can be mounted on or supported by a support substrate 110. The lid 120 can also be mounted to the support substrate and/or the semiconductor microchip to form the microfluidic chamber 130 between the various structures. A seal 150 can be applied between structures to bond structures together around joints, or can be applied to multiple structures to provide spacing between structures to contribute to providing the microfluidic chamber (See FIGS. 1, 5, and 6). The semiconductor microchip can be bonded to the support substrate by an adhesive 135 or other bonding technology, as shown in FIGS. 1-3, and 5. In other examples, the semiconductor microchip can be overmolded by the support substrate, as shown in FIG. 6. Other bonding techniques that can be used include wire bonding, die bonding, flip chip mounting, surface mount interconnect bonding, or the like. With respect to the use of adhesive between the support substrate and the semiconductor microchip, or between the lid and the support substrate and/or the semiconductor microchip, the adhesive can be a curable adhesive, such as an electromagnetic radiation curable adhesive, a heat curable adhesive, a chemical curable adhesive, or the like. In other examples, any of these structures can be fused together by welding, e.g., laser welding, ultrasonic welding, thermosonic welding, etc.

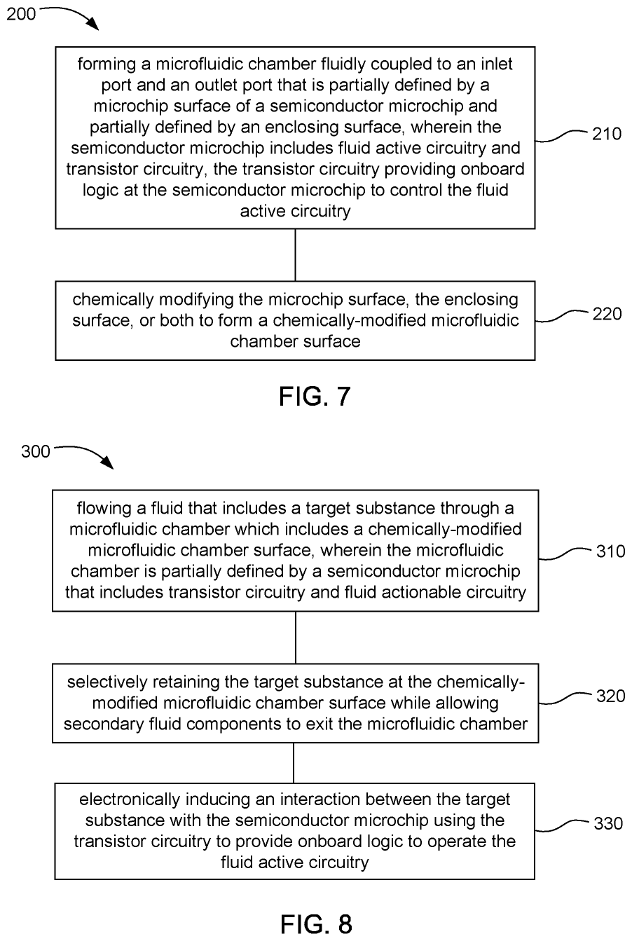

In accordance with other examples, as shown in FIG. 7, a method 200 of making a microfluidic device includes forming 210 a microfluidic chamber fluidly coupled to an inlet port and an outlet port that is partially defined by a microchip surface of a semiconductor microchip and partially defined by an enclosing surface, wherein the semiconductor microchip includes fluid active circuitry and transistor circuitry. The transistor circuitry in this example provides onboard logic at the semiconductor microchip to control the fluid active circuitry. The method further includes chemically modifying 220 the microchip surface, the enclosing surface, or both to form a chemically-modified microfluidic chamber surface. In one example, chemically modifying the microchip surface, the enclosing surface, or both can occur prior to assembly of the microfluidic chamber. In another example, chemically modifying the microchip surface, the enclosing surface, or both can occur after assembly of the microfluidic chamber.

In another example, as shown in FIG. 8, a method of electronically interacting with a target substance of a fluid includes flowing 310 a fluid that includes a target substance through a microfluidic chamber which includes a chemically-modified microfluidic chamber surface, wherein the microfluidic chamber is partially defined by a semiconductor microchip that includes transistor circuitry and fluid actionable circuitry. The method further includes selectively retaining 320 the target substance at the chemically-modified microfluidic chamber surface while allowing secondary fluid components to exit the microfluidic chamber, and electronically inducing 330 an interaction between the target substance with the semiconductor microchip using the transistor circuitry to provide onboard logic to operate the fluid active circuitry. The target substance can, for example, be a nucleic acid, and the secondary fluid components can include lysed cellular debris. In further detail, the onboard logic can control multiple operations of the fluid active circuitry based on conditions within the microfluidic chamber by selecting operations from multiple alternatives.

In accordance with this and other methods, one potential application for which the microfluidic devices of the present disclosure can be useful is in the extraction of nucleic acids, and in some examples, further work can be done relative to these extracted nucleic acids, e.g., amplification, post amplification purification, e.g. PCR purification, fluid analysis, and/or downstream applications such as cellular transfection. For example, a nucleic acid or nucleic acid sequence can be attracted to surfaces of the chemically-modified microfluidic chamber surfaces, e.g., covalent attachment, electrostatic attraction, adsorption, etc., from one fluid feed, and then with the concentrated nucleic acids or nucleic acid sequences concentrated thereon, a different fluid could be flowed through that introduces another property, e.g., ionic strength, pH, concentrations, etc., to carry out the second action. Chaotropic and/or kosmotropic agents associated with the chemically-modified microfluidic chamber surfaces could be used to encourage the adsorption of the nucleic acids to a surface of the chemically-modified microfluidic chamber surfaces, for example. Wash buffers, in some examples, can be introduced to microfluidic chamber surfaces that may as a result become attractive to target substances that may be flowing or may be extractable from a specific fluid. Fluids could be introduced, such as master mixes, that could be used for nucleic acid amplification and heat provided by the fluid active circuitry, etc.

In other examples, the fluid introduced into the microfluidic chamber can include lysed cells, which could be used for nucleic acids for amplification, antibody capture antibody, or the like. DNA or RNA could be purified, extracted, or otherwise separated from other cell components for amplification or for other purposes. For example, a sample that may include nucleic acids or proteins, such as from urine, blood, a swab, a plant sample, or the like, could be provided by lysing of cells, and then unwanted components or contaminants of the lysed cells could be cleared away, e.g., cell debris, blood cells (if not targeting blood samples), etc. Once the debris and other unwanted material has been passed downstream of the microfluidic chamber, an elution compound or buffer could be used to release the target component therefrom. In some instances, a wash buffer could be sent therethrough prior to the elution compound or buffer. Alternatively, it may be the target components of the fluid are not what is of interest, and they are captured so that they may be removed from the fluid, e.g., the chemically-modified microfluidic chamber surfaces may remove or be attracted to cell debris or unwanted chemical components, and the remaining fluid components could then be collected for use downstream from the microfluidic chamber. Either way, while in the microfluidic chamber, the fluid active circuitry of the semiconductor microchip may generate heat and provide sensor functions, e.g., electrochemical sensors, mixing, etc. For example, a sensor may be able to determine whether a sufficient amount of the debris and other chemical or biological components have been removed or are still present in preparation for carrying out the next function.

It is noted that, as used in this specification and the appended claims, the singular forms “a,” “an,” and “the” include plural referents unless the content clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and can be determined based on experience and the associated description herein.

As used herein, a plurality of items, structural elements, compositional elements, and/or materials may be presented in a common list for convenience. However, these lists should be construed as though members of the list is individually identified as a separate and unique member. Thus, no individual member of such list should be construed as a de facto equivalent of any other member of the same list solely based on their presentation in a common group without indications to the contrary.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if numerical values and sub-ranges is explicitly recited. For example, a weight ratio range of about 1 wt % to about 20 wt % should be interpreted to include not only the explicitly recited limits of 1 wt % and about 20 wt %, but also to include individual weights such as 2 wt %, 11 wt %, 14 wt %, and sub-ranges such as 10 wt % to 20 wt %, 5 wt % to 15 wt %, etc.

What is claimed is:

1. A microfluidic device, comprising:
a semiconductor microchip including fluid active circuitry and transistor circuitry, wherein the transistor circuitry provides onboard logic at the semiconductor microchip to control the fluid active circuitry; and
a microfluidic chamber fluidly coupled to an inlet port and an outlet port, wherein the microfluidic chamber is defined in part by a microchip surface with the active circuitry positioned to interact with fluid introduced into the microfluidic chamber and partially defined by an enclosing surface, wherein the microchip surface, the enclosing surface, or both is a chemically-modified microfluidic chamber surface that is selectively interactive with a target component of the fluid.

2. The microfluidic device of claim 1, wherein the microfluidic device further includes the fluid containing the target fluid, and the fluid is positioned within the microfluidic chamber.

3. The microfluidic device of claim 1, wherein the enclosing surface is provided by a lid including a material selected from glass, quartz, polymer, amorphous polymer, or a combination thereof.

4. The microfluidic device of claim 1, wherein the enclosing surface is provided by a support substrate that supports the semiconductor microchip, and the support substrate includes a material selected from metal, glass, silicon, silicon dioxide, ceramic, polyethylene, polypropylene, polycarbonate, poly(methyl methacrylate), epoxy molding compound, polyamide, liquid crystal polymer (LCP), polyphenylene sulfide, or a combination thereof.

5. The microfluidic device of claim 1, wherein the chemically-modified microfluidic chamber surface is modified with an antibody, streptavidin, an oligomer, an amine-containing functional group, a carboxyl-containing functional group, an organosilane, or a combination thereof.

6. The microfluidic device of claim 1, wherein the semiconductor microchip has an elongated aspect ratio with a width from 50 μm to 1 mm, a thickness from 50 μm to 1 mm, and a length of 1.5 mm to 50 mm, wherein the inlet port and the outlet port are positioned so that a flow of fluid therebetween is along the length of the semiconductor microchip.

7. The microfluidic device of claim 1, wherein the microfluidic chamber has a volume from 1 nL to 100 μL.

8. The microfluidic device of claim 1, wherein the fluid active circuitry includes a heater, a sensor, an electromagnetic radiation source, a fluid actuator, or a combination thereof.

9. A method of making a microfluidic device, comprising:
forming a microfluidic chamber fluidly coupled to an inlet port and an outlet port that is partially defined by a microchip surface of a semiconductor microchip and partially defined by an enclosing surface, wherein the semiconductor microchip includes fluid active circuitry and transistor circuitry, the transistor circuitry providing onboard logic at the semiconductor microchip to control the fluid active circuitry; and
chemically modifying the microchip surface, the enclosing surface, or both to form a chemically-modified microfluidic chamber surface.

10. The method of claim 9, wherein chemically modifying the microchip surface, the enclosing surface, or both occurs prior to assembly of the microfluidic chamber.

11. The method of claim 9, wherein chemically modifying the microchip surface, the enclosing surface, or both occurs after assembly of the microfluidic chamber.

12. A method of electronically interacting with a target substance of a fluid, comprising:
flowing a fluid that includes a target substance through a microfluidic chamber which includes a chemically-modified microfluidic chamber surface, wherein the microfluidic chamber is partially defined by a semiconductor microchip that includes transistor circuitry and fluid actionable circuitry;
selectively retaining the target substance at the chemically-modified microfluidic chamber surface while allowing secondary fluid components to exit the microfluidic chamber; and
electronically inducing an interaction between the target substance with the semiconductor microchip using the transistor circuitry to provide onboard logic to operate the fluid active circuitry.

13. The method of claim 13, wherein selectively retaining the target substance at the chemically-modified microfluidic chamber surface includes selectively retaining the target substance on two opposite facing major surfaces of the semiconductor microchip.

14. The method of claim 13, wherein the target substance is a nucleic acid, and the secondary fluid components include lysed cellular debris.

15. The method of claim 13, wherein the onboard logic controls multiple operations of the fluid active circuitry based on conditions within the microfluidic chamber by selecting operations from multiple alternatives.
